(19) United States
(12) Patent Application Publication
(10) Pub. No.: US 2024/0310347 A1
(43) Pub. Date: Sep. 19, 2024

(54) Systems and Methods for Determining the Sex of Plants

(71) Applicants: Mickal ADLER, Metarie, LA (US); John K. ROBERTS, III, Gretna, LA (US); James F. GUENTHER, Metarie, LA (US); Dzmitry KUROUSKI, College Station, TX (US)

(72) Inventors: Mickal ADLER, Metarie, LA (US); John K. ROBERTS, III, Gretna, LA (US); James F. GUENTHER, Metarie, LA (US); Dzmitry KUROUSKI, College Station, TX (US)

(21) Appl. No.: 18/594,232

(22) Filed: Mar. 4, 2024

Related U.S. Application Data
(60) Provisional application No. 63/489,344, filed on Mar. 9, 2023.

(51) Int. Cl. G01N 33/00; A01G 7/00

(52) U.S. Cl. CPC G01N 33/0098; A01G 7/00

(57) ABSTRACT

Herein described are spectroscopy-based methods to identify chemical distinctions between male and female plants and utilize such distinctions to determine the sex of a plant at a very early stage of development.

### PRIORITY

This application claims the benefit of and priority to U.S. Prov. Appl. No. 63/489,344, filed on Mar. 9, 2023, the entire contents of which are incorporated by reference herein.

### FIELD OF THE PRESENT INVENTION

The present invention relates generally to methods for determining the sex of plants, particularly Cannabis plants. More particularly, the present invention relates to the use of spectroscopic methods to identify chemical distinctions between male and female plants and utilize such distinctions to determine the sex of the plant at a very early stage of development.

### BACKGROUND OF THE PRESENT INVENTION

Like animals, certain plants are capable of sexual reproduction. Some plants, known as dioecious species, have separate male and female plants. One type of dioecious plant is the Cannabis plant. For certain species of dioecious plants it is useful to determine the sex of the plants at some point during their life cycle. For example, female Cannabis plants are more valuable than male Cannabis plants because their flowers, which only appear on the female plant, are enriched in cannabinoids and hence have significant commercial utility.

Male and female plants of some species, including Cannabis, can be distinguished by visual inspection. However, this method requires one to wait until the plants are somewhat mature, which means it can be weeks or months before a conclusive determination of sex can be made. In the interim, resources such as labor, fertilizer, and space are needlessly wasted on plants that have little commercial value. In addition, visual inspection is labor intensive and requires some skill on the part of the person doing the inspection, which adds to the cost and availability of this classification method.

Analysis of plant DNA (genetic screening) can also be used to determine the sex of plants [1]. Such screening methods generally involve the steps of (i) collecting a plant sample, (ii) preparing it for laboratory analysis, and then (iii) having it analyzed. This final step typically involves sending the plant sample offsite to a laboratory staffed with people of appropriate skill level and instruments capable of determining the sex of plants. As such, this process can be both slow and expensive, particularly when there are hundreds or thousands of plants to categorize.

Recently, a commercial unit for sexing Cannabis plants in the field has come on the market [1]. However, this unit, the “Gene-Up® Pro Gender ID” from bioMerieux, Inc. (Durham, NC), still requires the taking of a physical sample from a Cannabis plant coupled with a 5 minute sample preparation that involves extraction and pipetting followed by placement of the sample in a machine for 2 hours. Thus, while this device allows Cannabis growers to sex their plants themselves, it nevertheless is still slow, expensive, and takes skill on the part of the user.

Accordingly, given the drawbacks of the commercially available options noted above, there remains a consistent need in the art for a rapid and inexpensive yet precise method for differentiating male from female plants, particularly male from female Cannabis plants, at the earliest stages of development.

### SUMMARY OF THE PRESENT INVENTION

Spectroscopy is the study of the interaction of electromagnetic radiation with matter [2]. Spectroscopy has been used for decades to perform chemical analyses for varied applications, including, for example, the analysis of plant material. For example, near infrared spectroscopy has been known to be used to determine fat, protein, and moisture in a number of plant matrices [3]; likewise, Raman spectroscopy has been used to analyze Cannabis plants [4,5]. Compared to other chemical analysis techniques, spectroscopic techniques are often times faster, easier, and cheaper because, for example, they may require little or no sample preparation, and may be used in the field. For example, recent advances in spectroscopy have given rise to handheld instruments that are easy to use and require little sample preparation [6].

Noting the advantages of spectroscopy as a chemical analysis tool, certain skilled artisans have applied it to the problem of sexing plants. For example, in recent work Tormena et al [7] and Khan et. al [8] describe the study of yerba mate and date palm plants, respectively, using infrared spectroscopy, more particularly the discovery of a spectral difference between the extracts of male and female plants. Critically, in the context of the Tormena and Khan studies, the extracts of the male and female plants were shown to be chemically different. If this were not the case, the selected spectroscopic techniques would not have worked. To that end, because not all analytes give strong signals in all types of spectroscopic techniques, it is important to choose the right spectroscopic method for the analytes in question. For example, polar functional groups have strong infrared signals and weak Raman spectroscopy signals [9,10], whereas non-polar functional groups frequently have weak infrared signals and strong Raman spectroscopy signals [11, 12].

The problem with the spectroscopy extract methods described by Tormena and Khan[7,8], like the DNA method mentioned above, is that plant samples must be collected, taken back to a lab, processed properly, and then be analyzed. This is almost as slow, difficult, and expensive as DNA analysis. Ideally, there would be an accurate spectroscopic method for sexing plants that could be performed in the field with no sample preparation.

Fernandes et al. describe the use of near infrared (NIR) spectroscopy to study intact papaya seeds and leaves. However, Fernandes et al. made no study of the chemical differences between the different genders of papaya plants and hence had no idea whether they were picking the right spectroscopic technique, or indeed whether the proposed method would work at all, much less with the level of accuracy and precision required in the art. In addition, the method of Fernades suffers from a lack of knowledge of the chemical difference between plant genders, which results in there being no discernible spectral difference between the plant genders, and with this lack of knowledge, it becomes impossible to pick the right spectroscopic method for the job. It thus is no surprise that the Fernandes method performed poorly, with an at best 80% success rate in sexing papaya seeds and leaves [13].

Accordingly, there remains a need in the art for a spectroscopic method that can accurately sex plants in the field with no sample preparation. As evidenced by the present invention, the key to developing such a method is an understanding of the chemical differences between differently gendered plants so the best spectroscopy method for the analysis can be chosen. To wit, the recent work of Higgins, Jessup, and Kurouski discussed below has shown that male and female Cannabis plants contain differing amounts of the carotenoid lutein. Bearing in mind this chemical signature, the present invention is directed to the development of spectroscopic protocols that can serve to determine the sex of plants. Illustrative examples of such spectroscopic methods for determining the sex of plants, including Cannabis plants, are described in detail herein.

In the work of Higgins, Jessup, and Kurouski extracts of male and female hemp plants were analyzed by High Pressure Liquid Chromatography (HPLC). Leaves from hemp plants were collected, homogenized, and extracted with chloroform and methylene chloride. Leaf extracts were analyzed by reverse phase HPLC using a photodiode array detector (PDA) at 450 nm. The separation was accomplished using a C30, 3 micron diameter column using mobile phases of 95/5 v/v methanol/water (A) and tert-methyl butyl ether (B). The gradient elution used was 97% A and 3% B at 0-6 minutes with a linear increase of B to 100% at 20 minutes, and a return to initial conditions at 23 minutes. The column temperature was maintained at 20° C. Analysis of the results showed that female hemp plants contained statistically significantly more of the carotenoid lutein than male plants. The chemical structure of lutein is shown in FIG. 1.

Note the extended chain of conjugated C═C bonds in lutein. The intensity of Raman spectral peaks depends, amongst other things, on the polarizability of the chemical bonds in a functional group [11,12]. The electrons in the extended chain of conjugated C═C bonds in lutein are very polarizable, meaning lutein should give a strong Raman signal, and thus Raman spectroscopy is a good candidate for sexing Cannabis plants.

These results indicate spectroscopy may be used to measure the amount of lutein or other compounds in Cannabis plants and the results may be used to distinguish between male and female plants quickly, easily, and early on in the growth process. It is readily apparent to the present inventors that further identified chemical differences between male and female plants of other species may also be distinguished by spectroscopy. To that end, the work of Higgins, Jessup, and Kurouski showed that Raman spectra of hemp leaves displayed carotenoid peaks at 1115, 1155, 1185, 1218, and 1525 cm⁻¹. The size of these peaks is expected to respond to changes in carotenoid concentration, including changes in the concentration of lutein. Confirming this prediction, Raman spectra were successfully used to distinguish male from female hemp plants with 94% accuracy.

In further work, Goff et. al [5] used Raman spectroscopy to distinguish between male, female, and hermaphroditic Cannabis plants with better than 98% accuracy. This accuracy is significantly better than the NIR work of Fernandes et al. in sexing papaya plants [13]. Again, this is due to their lack of knowledge a priori of the chemical differences between papaya plant genders, and arbitrarily choosing near infrared spectroscopy rather than a type of spectroscopy that will give a strong signal for the analyte(s) of interest. The success of Higgins, Jessup, and Kurouski and Goff et al. [5] in sexing Cannabis plants confirm the inventive principle, namely that spectroscopic methods may be used to determine the sex of plants with a high degree of accuracy.

Thus, it is therefore an objective of the present invention to provide a method for determining the sex of a plant that involves the following steps:
(a) exposing a plant or plant part to a beam of light to allow the beam of light to interact with the plant or plant part,
(a) taking the beam of light after it has interacted with the plant or plant part and analyzing it with a spectral analyzer to determine a spectrum of the plant or plant part,
(a) applying a plurality of sexing algorithms to the spectrum to determine the sex of the plant (e.g., whether male, female or hermaphroditic), and
(a) communicating the sex determination to an output device.

It is further an objective of the present invention to apply the above method to the determination of the sex of the plant with a greater than 85% accuracy, preferably greater than 90%, 91%, 92%, 93%, 94%, or 95% accuracy, more preferably greater than 96%, 97%, 98%, or 99% accuracy.

In a particularly preferred embodiment, the plant is a Cannabis plant. However, in the context of the present invention, the plant part may be any of a root, stem, branch, leaf, flower, leaf, stem, or seed.

While the present invention is not limited to a particular spectroscopy method, in preferred embodiments, the spectroscopic method is selected from among radio wave, microwave, far infrared, mid-infrared, near infrared, visible, ultraviolet, x-ray, absorption, reflection, transmission, scattering, emission, and Raman. In a particularly preferred embodiment, the spectroscopy used is a Raman spectrum.

While the present invention is not limited to a particular of sexing algorithm, in preferred embodiments, the plurality of sexing algorithms may be selected from among principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, and library searching.

The present invention is not limited to a particular output device or connection. As such, the output device may be wired or wirelessly connected and, in preferred embodiments, can be selected from among a cellular phone, smart phone, and computer.

These and other aspects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims.

### BRIEF DESCRIPTION OF THE FIGURES

FIG. 1 presents the chemical structure of lutein.

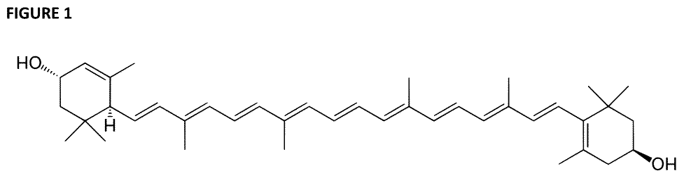

FIG. 2 presents the Raman spectrum of a Cannabis leaf.

FIG. 3 presents a flow chart for an embodiment of the present invention.

### DETAILED DESCRIPTION OF THE PREFERRED EMBODIMENTS

Unless otherwise defined herein, scientific and technical terms used in the present disclosure shall have the meanings that are commonly understood by those of ordinary skill in the art. In case of conflict, the present specification, including definitions, will control.

The term “a” or “an” entity refers to one or more of that entity; for example, “a vector,” is understood to represent one or more vectors.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless otherwise required by context, singular terms shall include pluralities and plural terms shall include the singular.

### Plants and Plant Parts:

Plants consist of several parts. The roots are typically found below soil level. The stem or trunk is typically a vertical member that grows up out of the ground. A plant branch is often an offshoot from a stem or trunk. A leaf is typically an offshoot from a stem or branch where significant photosynthetic activity takes place. A flower is a type of reproductive organ on a plant. A plant seed is the result of plant sexual reproduction which when planted produces a new plant.

For the purposes of the present invention, and as should be obvious to one of ordinary skill in the art, the terms “plant” and “plant part” are used interchangeably to refer to all parts of any plant including but not limited to roots, stems, trunk branches, flowers, and seeds.

Plants can be categorized as “male”, whose reproductive organs give off pollen, “female”, which are fertilized by the pollen, and “hermaphroditic”, i.e., plants containing both male and female reproductive organs. In the context of the present invention, the various forms of the verb “to sex” in relationship to plants refer to any process by which one determines the sex of the plant. In the context of the present invention, spectroscopic method for “sexing” plants, particularly Cannabis plants, are particularly preferred.

### Spectroscopy:

Spectroscopy uses electromagnetic radiation to analyze samples. For the purposes of the present invention, any type of electromagnetic radiation may be used including, but not limited to, radio waves, microwaves, far infrared, mid-infrared, near infrared, visible, ultraviolet, and x-rays.

Different types of electromagnetic radiation are defined by their wavelength, amongst other properties, which is discussed in the literature [2]. For the purposes of the present invention, the term “light” will encompass any and all types of electromagnetic radiation.

In the particle model of light, beams of electromagnetic radiation can be thought of as containing massless particles called photons [2]. When a beam of light interacts with a sample, many different types of phenomena can occur, examples of which include, but are not limited to, absorption, transmission, emission, reflection, refraction, diffraction, and scattering. The photons that have interacted with a sample can be collected and their properties determined. This is typically done with a spectral analyzer or “spectrometer”.

For the purposes of the present invention the types of spectral analyzer that can be used in the context of the inventive methods include, but are not limited to, dispersive, Fourier transform, non-dispersive, and Fabry-Perot.

For the purposes of the present invention the spectral properties that can be measured by a spectral analyzer include. but are not limited to. wavelength, wavenumber, frequency, intensity, absorbance, reflectance, reflectivity, transmittance, percent transmittance, emission, emissivity, scattering intensity, counts, Raman scattering intensity, and arbitrary intensity.

Spectroscopy requires a light source to work. For the purposes of the present invention, the types of light sources that may be used include, but are not limited to, broad band sources, narrow band sources, and lasers.

### Sexing Algorithms:

Once the spectrum of a plant sample is measured, it is convenient to use a computing device running an algorithm to determine the sex of the plant from its spectrum. These algorithms are referred to herein as “sexing algorithms”. For the purposes of the present invention, any set of mathematical algorithms that can be used to analyze a spectrum may constitute a sexing algorithm. Illustrative examples of sexing algorithms suitable for use in the context of the present invention include, but are not limited to, principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, and library searching.

In the context of the present invention, the term “plurality of sexing algorithms” means that any one of these algorithms may be used to sex plants, or, alternatively, two or more of them may be used in an analysis to sex plants.

### Raman Spectroscopy:

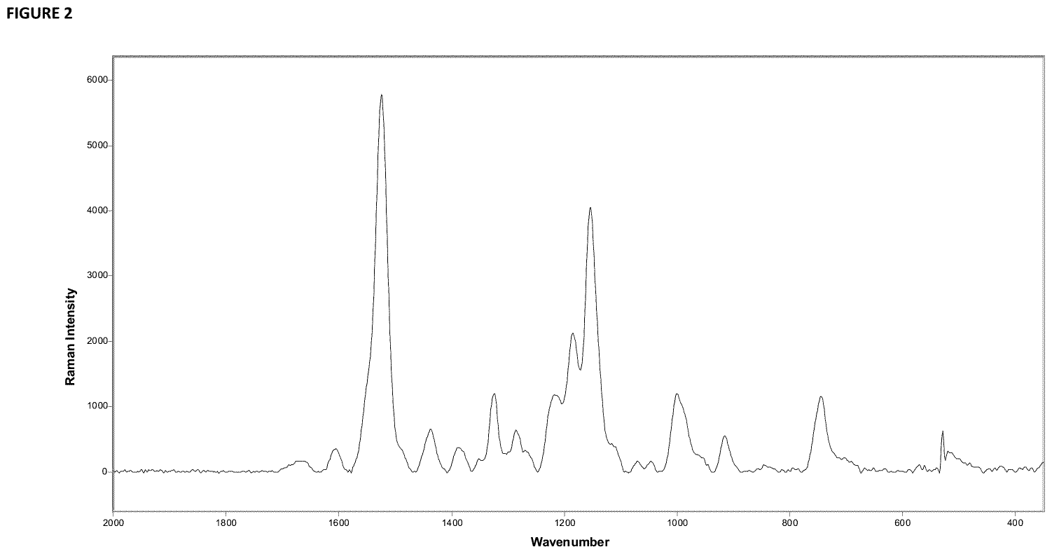

Raman scattering occurs when photons are inelastically scattered by molecules [peter, McCreery]. Because of the law of conservation of energy, the amount of energy lost by the inelastically scattered photons equals the amount of energy gained by the molecule from which it is scattered. Typically, the collision between a photon and a molecule excites vibrational modes of the molecule. For example, if a methyl (CH₃) group has a C—H stretch vibrational energy level at 2962 cm⁻¹, a photon of higher energy may collide with the molecule containing the methyl group, lose 2962 cm⁻¹ of energy and excite this vibration of the methyl group. In Raman spectroscopy, the inelastically scattered photons are gathered, analyzed by a spectral analyzer, and then plotted with appropriate units. When plotted with Raman Shift on the x-axis, the peak positions in a Raman spectrum represent the energy of vibrational energy levels excited. This gives chemical information which can be used to identify molecules and measure their concentrations in samples. The Raman spectrum of a Cannabis leaf is seen in FIG. 2.

In an embodiment of the present invention, Raman spectroscopy is used to sex plants. More particularly, Raman spectroscopy may be used to sex Cannabis plants.

### Results Reporting and Output Devices:

For a spectroscopy-based plant sexing method to be useful, the method needs to let the user know the results of the sexing analysis. The present invention contemplates the use of any type of output device capable of displaying text and graphics may be used. Thus, in the context of the present invention, illustrative examples of such output devices include, but are not limited to, cathode ray tube screens, liquid crystal displays, televisions, computer screens, cell phones, and smart phones.

In the context of the present invention, the output device is preferably configured to save an electronic copy of the spectroscopic results in any file format. Examples of such output device that capable of saving an electronic copy of the results include, but is not limited to, floppy disks, hard disks, USB drives, networks, network servers, and remote storage such as in the cloud. An output device may also provide a paper copy of the results. Examples of paper copy output devices contemplated by the present invention include plotters and printers. Output devices suitable for use in the inventive context may incorporate one, some, or all of the above capabilities.

In the context of the present invention, the spectral analyzer and output device may be part of one unit or they may be separate units. In either case, the results of the sexing algorithm analysis must be communicated to the output device. This can be done using a wired connection, examples of which include, but are not limited to, serial, parallel, USB, and Ethernet. Alternatively, the spectral analyzer and output device may communicate wirelessly. Examples of wireless protocols that may be used in the context of the present invention include, but are not limited to, Wi-Fi and Bluetooth.

### Cannabis Plants:

Human beings have been growing and using Cannabis plants for thousands of years. For the purposes of the present invention, the term “Cannabis plants” broadly encompasses all parts of the plant of species Cannabis Indica, Cannabis Sativa, and Cannabis Ruderalis, for example.

As noted above, Cannabis plants are dioecious, meaning they exist in male and female forms. Some Cannabis plants may also be hermaphroditic, meaning they contain male and female reproductive organs. Amongst the many useful compounds contained in Cannabis plants are cannabinoids, such as Δ-9 Tetrahydrocannabinol (THC) and cannabidiol (CBD). These compounds have medicinal value, and THC is known to be psychotropic. Cannabinoids occur in high concentrations in the flowers of Cannabis plants, which are found on the female plants. Male Cannabis plants typically do not have flowers and typically have lower concentrations of cannabinoids than the flowers of female Cannabis plants. Hence, the flowers of female Cannabis plants have significant commercial value.

In Cannabis cultivation, given the absence of flowers and lower concentrations of cannabinoids, male plants are weeded out because they have little economic value and thus female plants are grown preferentially. Currently, when a Cannabis seed is planted, growers typically do not know what sex the plant will be. For visual inspection to be able to distinguish between male and female Cannabis plants, it takes several weeks of growth before it is obvious whether the plant bears flowers or not. This means a significant amount of time, energy, money, and resources is spent growing plants of little economic value (i.e., male plants). So as to avoid wasting time, energy, money, and resources cultivating unwanted male plants, it would be highly desirable for there to be a method for sexing Cannabis plants earlier in the growth cycle, say after a few days. The present invention addresses this need in the art by providing a rapid and inexpensive yet precise method for determining the sex of a plant, particularly for differentiating male from female Cannabis plants, at the earliest stages of development.

### ILLUSTRATIVE EMBODIMENTS

Hereinafter, the present invention is described in more detail by reference to certain examples and preferred embodiments. However, it should be obvious to anyone of ordinary skill in the art that the details mentioned in all embodiments are for illustrative purposes only, and that many other variations of the present invention are possible while still being well within the scope of the present invention. As such, the following embodiments are meant to be enabling and for illustrative purposes only and are not meant to narrow the scope of the present invention in any fashion whatsoever. As such, methods and materials similar or equivalent to those described herein can be used in the practice or testing of the present invention.

### Example 1

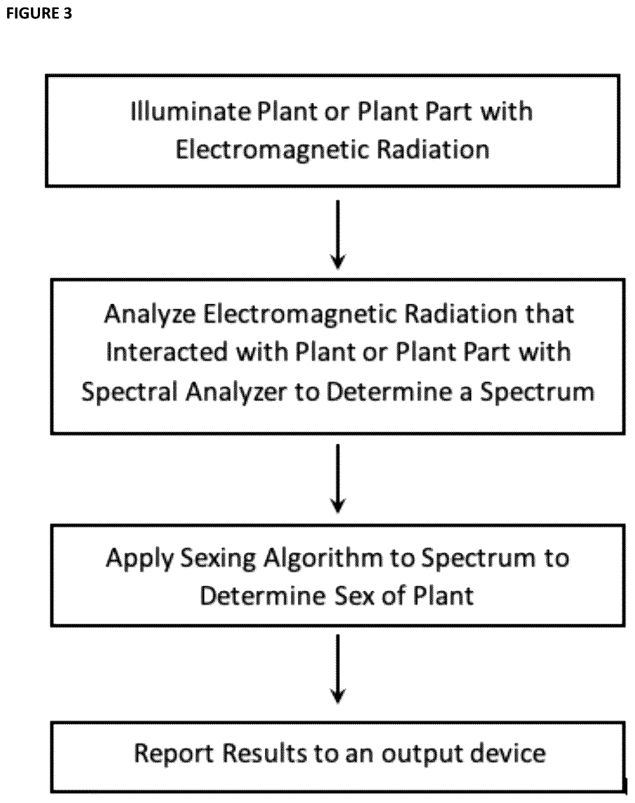

In an exemplary embodiment, a laser of wavelength 831 nm and 495 milliwatts power is caused to illuminate the adaxial side of a Cannabis leaf for 1 second. The scattered photons are analyzed and a Raman spectrum is measured using an Agilent Resolve handheld Raman Spectrometer. The measured Raman spectrum is analyzed using a partial least squares discriminant analysis sexing algorithm, and the results reported wirelessly to a cellular phone. The flow chart of FIG. 3 provides a graphical representation of these steps.

### REFERENCES

All publications, patent applications, patents and other references mentioned herein are incorporated by reference in their entirety.
[1] bioMerieux “Gene-Up® Pro Gender ID”, www.biomerieux.com
[2] Brian C. Smith, Fundamentals of Fourier Transform Infrared Spectroscopy 2^{nd} Ed., CRC Press, Boca Raton, 2011.
[3] D. Burns and E. Ciurczak, Handbook of Near Infrared Analysis, Marcel Dekker, New York, 1992.
[4] L. Sanchez, D. Baltensperger, and W. Kurouski, Anal. Chem., 92: 7733 (2020).
[5] N. Goff, J. Guenther, J. K. Roberts, M. Adler, M. Dalle Molle, G. Matthews, and D. Kurouski, Molecules 27: 4978 (2022).
[6] www.mariposatechnology.com
[7] C. Tormena et al., Microchemical Journal, 158: 105329 (2020).
[8] A. Khan et al., Plants, 10: 536 (2021).
[9] D. Tao et al., Spetrochimica Acta Part A: Molecular and Biomolecular Spectroscopy, 2008: 7-12 (2019).
[10] E. Ciurczak, B. Igne, J. Workman, and D. Burns Eds., Handbook of Near-Infrared Analysis, CRC Press, Boca Raton, 2021.
[11] P. Larkin, IR and Raman Spectroscopy Principles and Spectral Interpretation, Elsevier, Boston, 2011.
[12] Richard L. McCreery, Raman Spectroscopy for Chemical Analysis, Wiley, New York, 2000.
[13] T. Fernandes et al., Computers and Electronics in Agriculture, 193: 106674 (2022).
[14] S. Higgins, R. Jessup, and D. Kurouski, Planta 85: 255 (2022).

While the invention has been described in detail and with reference to specific embodiments thereof, it will be apparent to one skilled in the art that various changes and modifications can be made therein without departing from the spirit and scope of the invention.

What is claimed:

1. A method for determining the sex of a plant, said method comprising the following steps:
a. first, exposing a plant or plant part to a beam of light to allow said beam of light to interact with said plant or plant part,
b. next, a taking said beam of light after it has interacted with said plant or plant part and analyzing it with a spectral analyzer to determine a spectrum of said plant or plant part,
c. then, applying a plurality of sexing algorithms to said spectrum to determine the sex of said plant, and
d. optionally, communicating said sex determination to an output device.

2. The method of claim 1, wherein said plant part is selected from the group consisting of: a root, stem, branch, leaf, flower, leaf, stem, and seed.

3. The method of claim 1, wherein the type of spectroscopy used is selected from the group consisting of: radio wave, microwave, far infrared, mid-infrared, near infrared, visible, ultraviolet, x-ray, absorption, reflection, transmission, scattering, emission, and Raman.

4. The method of claim 1, wherein the plurality of sexing algorithms used is selected from the group consisting of: the list principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, and library searching.

5. The method of claim 1, wherein the results of the sexing algorithm are communicated to an output device via a wired or wireless connection.

6. The method of claim 1, wherein the output device is selected from the group consisting of: a cellular phone, smart phone, and computer.

7. The method of claim 1, wherein the sex of the plant to be determined is male, female, or hermaphroditic.

8. A method for determining the sex of a Cannabis plant, said method comprising the steps of:
a. first, exposing a Cannabis plant or plant part to a beam of light to allow said beam of light to interact with said Cannabis plant or plant part,
b. second, taking said beam of light after it has interacted with said Cannabis plant or plant part and analyzing it with a spectral analyzer to determine a spectrum of said plant or plant part,
c. third, applying a plurality of sexing algorithms to said spectrum to determine the sex of said plant, and
d. optionally, communicating said sex determination result to an output device.

9. The method of claim 8, wherein said plant part is selected from the group consisting of: a root, stem, branch, leaf, flower, leaf, stem, and seed.

10. The method of claim 8, wherein the type of spectroscopy used is selected from the group consisting of: radio wave, microwave, far infrared, mid-infrared, near infrared, visible, ultraviolet, x-ray, absorption, reflection, transmission, scattering, emission, and Raman.

11. The method of claim 8, wherein the plurality of sexing algorithms used is selected from the group consisting of: principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, and library searching.

12. The method of claim 8, wherein the results of the sexing algorithm are communicated to an output device via a wired or wireless connection.

13. The method of claim 8, wherein the output device is selected from the group consisting of: a cellular phone, smart phone, and computer.

14. The method of claim 8, wherein the sex of the plant to be determined is male, female, or hermaphroditic.

15. A method for determining the sex of a Cannabis plant, said method comprising the steps of:
a. first, exposing a Cannabis plant or plant part to a beam of light to allow said beam of light to interact with said Cannabis plant or plant part,
b. second, taking said beam of light after it has interacted with said Cannabis plant or plant part and analyzing it with a spectral analyzer to determine a Raman spectrum of said plant or plant part,
c. third, applying a plurality of sexing algorithms to said Raman spectrum to determine the sex of said plant, and
d. finally, communicating said sex determination results to an output device.

16. The method of claim 15, wherein said plant part is selected from the group consisting of: a root, stem, branch, leaf, flower, leaf, stem, and seed.

17. The method of claim 15 wherein the plurality of sexing algorithms used is selected from the group consisting of: principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, and library searching.

18. The method of claim 15, wherein the results of the sexing algorithm are communicated to an output device via a wired or wireless connection.

19. The method of claim 15, wherein the output device is selected from the group consisting of: a cellular phone, smart phone, and computer.

20. The method of claim 15, wherein the sex of the plant to be determined is male, female, or hermaphroditic.
